(19) United States
(12) Patent Application Publication
(10) Pub. No.: US 2016/0265032 A1
(43) Pub. Date: Sep. 15, 2016

(54) PORTABLE DNA ANALYSIS MACHINE

(71) Applicant: Hexna Corp., Windermere, FL (US)

(72) Inventors: Avishaan SETHI, San Francisco, CA (US); Harjus Sethi, West Chester, OH (US)

(21) Appl. No.: 15/068,050

(22) Filed: Mar. 11, 2016

Related U.S. Application Data
(60) Provisional application No. 62/131,544, filed on Mar. 11, 2015.

(51) Int. Cl. C12Q 1/68; B01L 7/00

(52) U.S. Cl. CPC C12Q 1/686; B01L 7/52; B01L 2200/10; B01L 2300/021; B01L 2300/0627; B01L 2300/18; B01L 2300/023; B01L 2300/0861

(57) ABSTRACT

Aspects of the present disclosure relate to DNA amplification systems, e.g., PCR chips used to hold a testing solution and sample, and systems and methods for testing the sample including thermocycling and detection. The system can include a containment chip capable of holding a sample and reagents for amplifying a nucleic acid in the sample and containing a unique identifier used to choose a nucleic acid amplification protocol. The system can also include a nucleic acid detection component which may contain a light source, a light sensor, a temperature control unit and a processor.

### CROSS REFERENCE TO RELATED APPLICATIONS

This application claims priority to U.S. Provisional Application No. 62/131,544, filed Mar. 11, 2015, entitled “Portable DNA Analysis Machine,” the contents of which is incorporated herein in its entirety.

### BACKGROUND

1. Field

The present disclosure relates to the field of portable deoxyribonucleic acid (“DNA”) analysis. More specifically, the disclosure relates to a method and device for a highly portable DNA amplification device, e.g., a polymerase chain reaction (“PCR”) based system including an onboard CPU, light source, light source detector, and thermal cycler/heating element. The system can analyze tube and/or chip based DNA samples. The system has the flexibility to perform non-PCR tests as well.

2. Description of the Related Art

PCR is a biochemical technology in molecular biology used to amplify pieces of DNA across several orders of magnitude, generating thousands to millions of copies of a particular DNA sequence. The test is used for DNA replication and identification.

Though the principles of PCR can be straight forward, conducting the test can be difficult. Conventional PCR systems involve costly laboratory equipment to perform extended procedures, and require skilled scientists. The applications for PCR are widespread and the contexts for its use are diverse. Industries in medicine, forensics, biology, agriculture, food science, environmental science, archeology, anthropology, and others can all benefit from PCR. The procedure is especially effective in disease, tissue, and organism detection.

### BRIEF SUMMARY

Aspects of the present disclosure relate to DNA amplification systems, e.g., PCR chips used to hold a testing solution and sample, and systems and methods for testing the sample including thermocycling and detection. The described device can be capable of performing tag-polymerase PCR and Assembly PCR. In some embodiments, a DNA detection method is based in part on light fluorescence based detection methodology. In some embodiments, a DNA detection method is based in part on light absorption detection methodology. In some embodiments, a DNA detection method is based in part on the measurement of the change of impedance and/or capacitance indirectly via voltage measurements of a sample. In some embodiments, a containment chip containing electrodes allowing for impedance measurements of the sample is provided. In some embodiments, a containment chip with a clear pass through allowing the sample to both absorb as well as pass through or transmit light to a detector for DNA detection is provided. In some embodiments, a heating element is provided, which allows the temperature of the DNA sample to be increased, either directly or indirectly. In some embodiments, a cooling element is provided, allowing the DNA sample, either directly or indirectly, to have its temperature decreased. In some embodiments, a temperature sensing mechanism can be used to either directly or indirectly measure the temperature of the sample using either contact or non-contact measurement techniques. In some embodiments, a real-time monitoring method allows the sample to be tested during, outside, or in between the thermocycling process of the specification/protocol. In some embodiments, a method of processing the data outside of the system via either wireless or wired link allowing for the control and analysis to be done on external computing hardware is provided.

In one aspect, the present disclosure relates to a method for testing a DNA sample on a containment chip within a DNA analysis module, the method including: electronically transmitting a request for a testing protocol corresponding to the DNA sample to a remote server, the testing protocol including a thermocycling sequence; electronically receiving the testing protocol from the remote server; calibrating the DNA analysis module based on the testing protocol; initiating the thermocycling sequence according to the testing protocol; monitoring the temperature of the containment chip throughout the thermocycling sequence; adjusting the temperature of the containment chip to maintain the temperature of the containment chip within one or more temperature boundaries of the testing protocol; and periodically checking the DNA sample to determine if a positive result is obtained; if a positive result is obtained, ending the thermocycling sequence early; if a positive result is not obtained, continuing the thermocycling sequence.

In another aspect, the present disclosure relates to a DNA module, including: a containment chip, wherein the containment chip can include a DNA sample and an identifier for uniquely identifying the DNA sample; an optical reader for reading the unique identifier from the containment chip; one or more temperature control components for controlling the temperature of the containment chip; one or more DNA detection components for detecting one or more attributes of the DNA sample while the DNA analysis module performs a thermocycling process on the DNA sample; a wireless transmitter, capable of both downloading and uploading data; a processor configured to perform the following steps: receive the unique identifier from the optical reader; electronically transmitting, using the wireless transmitter, the unique identifier, to a remote server; electronically receiving a testing protocol from the remote server, wherein the testing protocol corresponds to the unique identifier; initiating the thermocycling sequence according to the testing protocol; monitoring, using the one or more temperature control components, the temperature of the containment chip throughout the thermocycling sequence; adjusting, using the temperature control components, the temperature of the containment chip to maintain the temperature of the containment chip within one or more temperature boundaries of the testing protocol; and periodically checking, using the DNA detection components, the DNA sample to determine if a positive result is obtained; if a positive result is obtained, ending the thermocycling sequence early; if a positive result is not obtained, continuing the thermocycling sequence.

In one aspect the present disclosure relates to a nucleic acid amplification system comprising a containment chip capable of holding a first sample wherein the containment chip comprises at least one reagent for amplifying a nucleic acid in the sample and an identifier for uniquely identifying the containment chip, wherein the identifier is used to select a nucleic acid amplification protocol, a nucleic acid detection component comprising a light source operably linked to the containment chip, wherein the light source provides light at the wavelength necessary to perform either absorption or fluorescence measurements of the sample, a light sensor operably linked to the containment chip, wherein the light sensor measures either absorption or fluorescence from the containment chip, a temperature control unit operably linked to the containment chip, wherein the temperature control unit measures the temperature of the sample, and adjusts the temperature of the containment chip, and a processor operably linked to the nucleic acid detection component and the temperature control unit, wherein the processor adjusts the light source, receives data from the light sensor, receives data from the temperature control unit, and adjusts the temperature, wherein the processer is operably connected to a database which contains the nucleic acid amplification protocol. In one embodiment, of the nucleic acid amplification system the nucleic acid detection component comprises an impedance detector operably linked to the containment chip. In another embodiment of the nucleic acid amplification system the nucleic acid detection component comprises a capacitance detector operably linked to the containment chip. In another embodiment of the nucleic acid amplification system a computing unit is in communication with the processor. In another embodiment of the nucleic acid amplification system the light source comprises one of a laser, a light emitting diode, an electroluminescence wire, an electroluminescence panel, a fluorescent light source, a tungsten lamp, a halogen lamp, a liquid crystal display screen, a silicon nitride lamp, a krypton lamp, a deuterium lamp, a sodium vapor lamp, a mercury vapor lamp, or a xenon light source. In another embodiment of the nucleic acid amplification system a temperature control unit comprises one of a heating element or a cooling element. In another embodiment of the nucleic acid amplification system the nucleic acid detection component further comprises spaced electrical contacts for applying an electrical current to the sample. In another embodiment of the nucleic acid amplification system the containment chip further comprises an optical filter allowing the sample to both absorb as well as pass through or transmit light to the detector. In another embodiment of the nucleic acid amplification system the containment chip comprises a sub-containment chip capable of holding reagents for nucleic acid amplification; and a second optical filter. In another embodiment of the nucleic acid amplification system the nucleic acid sample is one of a DNA sample or an RNA sample. In another embodiment of the nucleic acid amplification system the identifier for uniquely identifying the nucleic acid sample comprises one of a bar code a QR code, or an RFID. In another embodiment of the nucleic acid amplification system the containment chip is capable of holding a plurality of samples. In another embodiment of the nucleic acid amplification system the containment chip further comprises a second optical filter capable of blocking light of a specific wavelength. In another embodiment of the nucleic acid amplification system the containment chip further comprises a plastic film capable of being peeling back and resealed.

Another aspect of the disclosure includes a method of analyzing a nucleic acid sample using a nucleic acid detection system comprising providing a nucleic acid sample in a containment chip wherein the containment chip comprises at least one reagent for amplifying a nucleic acid in the nucleic acid sample and an identifier for uniquely identifying the containment chip, obtaining a nucleic acid amplification protocol from a database based on the unique identifier on the containment chip, and performing the steps of the nucleic acid amplification protocol. In one embodiment of the method the identifier for uniquely identifying the nucleic acid sample comprises one of a bar code, a QR code or an RFID. In another embodiment the method further comprises the step of monitoring nucleic acid amplification, wherein the nucleic acid amplification protocol can be stopped once nucleic acid amplification is detected.

Another aspect of the disclosure includes a method of analyzing nucleic acid using a nucleic acid detection system comprising providing a nucleic acid sample in a containment chip wherein the containment chip comprises at least one reagent for amplifying a nucleic acid in the sample and an identifier for uniquely identifying the containment chip, obtaining a nucleic acid amplification protocol from a database based on the unique identifier on the containment chip, performing a calibration step, comprising illuminating a light source, detecting light from a light sensor for calibration, and interpreting, by a processor in order to set a baseline, performing an initial test to determine if a positive result is obtained by comparing to the baseline, beginning a thermocycling sequence if a positive result is not obtained during the initial test, maintaining the nucleic acid sample within temperature boundaries defined by the nucleic acid amplification protocol using a temperature control unit, performing a test to determine if a positive result is obtained by comparing to the baseline, continuing thermocycling if a positive result is not obtained during the test, and ending thermocycling if a positive result is obtained during the test. In one embodiment the method includes the step of performing a test to determine if a positive result is obtained occurs during, outside, or in between the thermocycling process. In another embodiment the method further comprises the step of electronically transmitting a request for a testing protocol corresponding to a sample to a remote server.

### BRIEF DESCRIPTION OF THE DRAWINGS

Various objects, features, and advantages of the present disclosure can be more fully appreciated with reference to the following detailed description when considered in connection with the following drawings, in which like reference numerals identify like elements. The following drawings are for the purpose of illustration only and are not intended to be limiting.

FIG. 1 is a schematic component diagram of a DNA analysis module, according to certain embodiments of the present disclosure.

FIG. 2 is a block diagram of another exemplary top view layout of DNA analysis module, according to certain embodiments of the present disclosure.

FIG. 3A is a top view of a containment chip, according to certain embodiments of the present disclosure.

FIG. 3B is a side view of a containment chip, according to certain embodiments of the present disclosure.

FIG. 4 is an exemplary flow chart of a process of analyzing DNA, according to certain embodiments of the present disclosure.

### DETAILED DESCRIPTION

A traditional PCR method is comprised of two stages: thermocycling and electrophoresis. The first stage, thermocycling, consists of twenty to forty repeated temperature cycles, which each consist of two to three temperature changes for a specific length of time. Conventional PCR testing can require the scientist to calibrate a variety of parameters including the temperature, time, and number of cycles. Further, each parameter can be dependent on various other parameters such as the enzyme used, concentration level of the DNA, the type of DNA being tested, the melting temperature of the primers, and concentration of the divalent ions and deoxyribonucleoside triphosphates (“dNTPs”). The steps typically include: initialization, denaturation, annealing, extension, elongation, and final hold.

After the thermocycling is complete, the scientist then can perform gel electrophoresis in order to pseudo-quantify the amount of targeted DNA in the sample. Though conventional gel electrophoresis is inaccurate for quantification purposes, it is accurate in detecting positive DNA matches.

While traditional PCR systems are costly and complicated, the present implementation is founded on ease of use, compactness, high speed, and low cost. This is accomplished in part by integrating the device with a database of protocols, for example, open source protocols, allowing the user to select and perform a test. This will allow a user to quickly setup the device, download a testing protocol, and run a test from non-laboratory environments. The compactness and cost of the device allows a non-laboratory user to easily and inexpensively acquire the device. The compact device according to certain embodiments will not run more than ten samples at a time, whereas a traditional system generally is much larger in order to run hundreds of samples at a time. In some embodiments, the device can run more than ten samples, for example, 15-20 samples, or up to 50 samples. By only running a limited number of samples and using standardized electronic components, the cost of PCR/DNA analysis can be significantly reduced and accessibility improved.

Further enhancements according to certain embodiments include: a fluorescence detection method for measuring DNA products using either a charged coupled device (“CCD”) or light sensor, real-time DNA products monitoring during the thermocycling phase of the DNA analysis cycle, and UV light analysis in the quantification of PCR products.

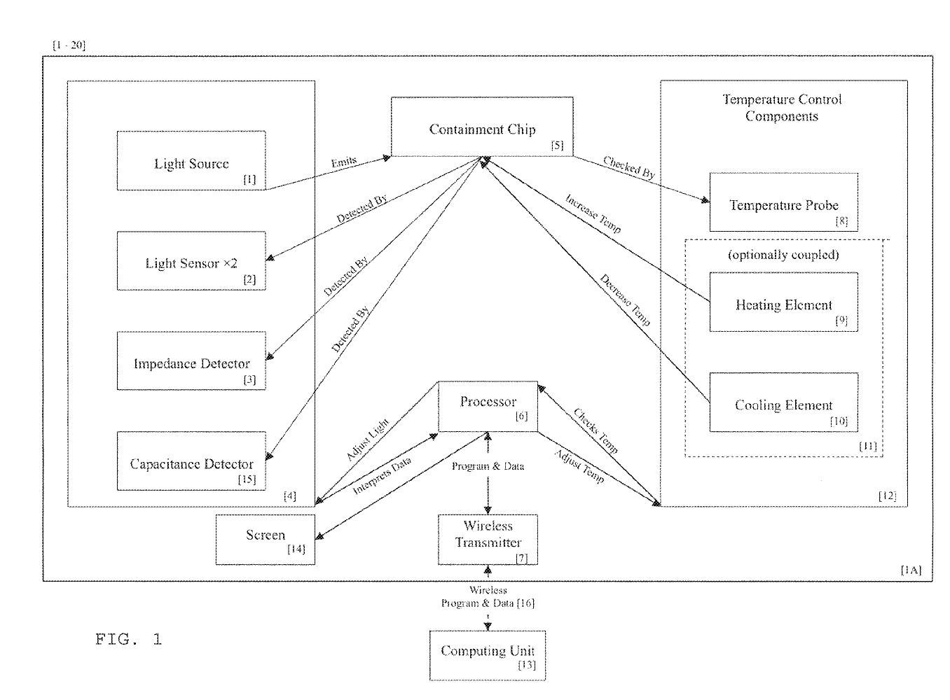

FIG. 1 is a schematic component diagram of a DNA analysis module 1A, according to aspects of the present disclosure. DNA analysis module 1A can include DNA detection components 4, including a light source 1, a light sensor 2, and an impedance detector 3; a containment chip 5; a processor 6; a wireless transmitter 7; temperature control components 12, including a temperature probe 8, and a heating element 9 and a cooling element 10; a screen 14, and a capacitance detector 15. DNA analysis module 1A can send data and programs 16 wirelessly to a connected computing unit 13.

DNA detection components 4 are a logical set of components directly or indirectly responsible for measuring the amount of DNA in the sample on the containment chip 5.

Light source 1 provides light at the wavelength necessary to perform either absorption or fluorescence measurement of the sample as per the experimental protocol. Accordingly, light source 1 emits light directed to the containment chip 5. Light source 1 can be a basic/multiline/tunable lasers, a light emitting diode (“LED”), an electroluminescence wire/panel, a fluorescent light source, a tungsten lamp, a halogen lamp, a liquid crystal display (“LCD”) screen, one of a silicon nitride, krypton, deuterium, or sodium vapor lamp, a mercury vapor lamp, a xenon light source or any other light sources emitting in the infrared to ultraviolet spectrum not listed here.

Light sensor 2 can be used for measuring absorption or fluorescence from the containment chip 5. In the case of measuring absorption, it can detect the absorption of a sample. In the case of measuring fluorescence, it can detect the fluorescence by the sample at the wavelength described in the experimental protocol, either with or without light wavelength filters. Light sensor 2 can be a used for absorbance, reflectance, transmission, fluorescence. Light sensor 2 can be a microspectrometer, a laser light sensor, an acoustic light sensor, a Golay sensor, a colorimeter, a photoresistor, a frequency based spectroanalyser, a charge coupled device (“CCD”), a camera based photo sensor, or a complementary metal oxide semiconductor (“CMOS”) sensor.

Impedance detector 3 can be used for measuring the change in electrical impedance of the sample in containment chip 5. Impedance detector 3 can be any sensor or detector that will measure the resistance, in ohms, of a sample.

Capacitance detector 15 can be used for measuring the change in electrical capacitance of the sample in containment chip 5. Capacitance detector 15 can be any sensor or detector that will measure the capacitance of the sample such as the PICOCAP® (Acam-messelectronic GMBH, Germany) capacitance measuring sensor.

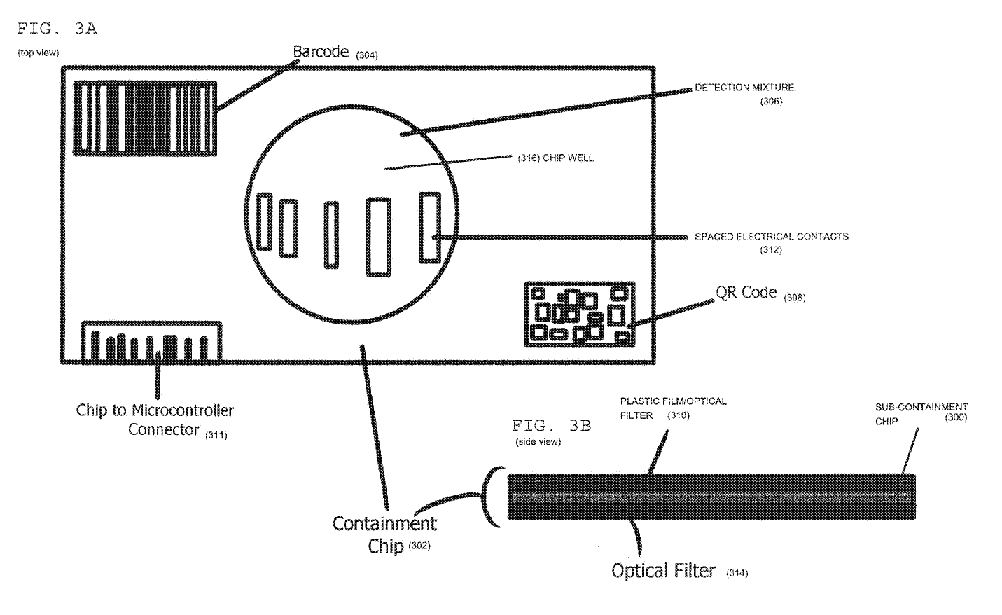

Containment chip 5 can be a pre-manufactured containment chip. Containment chip 5 allows the user to deposit a sample with the appropriate primers and testing solution. Containment chip 5 is then sealed and inserted into the machine. Containment chip 5 can be manufactured with a coating allowing it to filter specific wavelengths of light in order to allow detection of specific wavelengths of light as per the testing protocol. Containment chip 5 can have a label showing the unique testing protocol ID along with a QR code. Containment chip 5 receives light from light source 1, transmits light to light sensor 2, and the temperature of containment chip 5 can be monitored by temperature control components 12. Containment chip 5 interfaces with impedance detector 3 and capacitance detector 15 via spaced electrical contacts located on the containment chip (item 312 pictured in FIG. 3A). Containment chip 5 is described in more detail with respect to FIG. 3A and FIG. 3B.

Processor 6 can control DNA detection component 4, receive information from DNA detection component 4, receive information from temperature control component 12, send data to wireless transmitter 7, and receive data and program protocol information from wireless transmitter 7. Processor 6 can adjust light from light source 1, interpret data from DNA detection components 4, provide rendering to screen 14, transmit data to wireless transmitter 7, receive a protocol from wireless transmitter 7, check temperature of the sample from temperature control components 12, and adjust the temperature of sample through control of temperature control components 12.

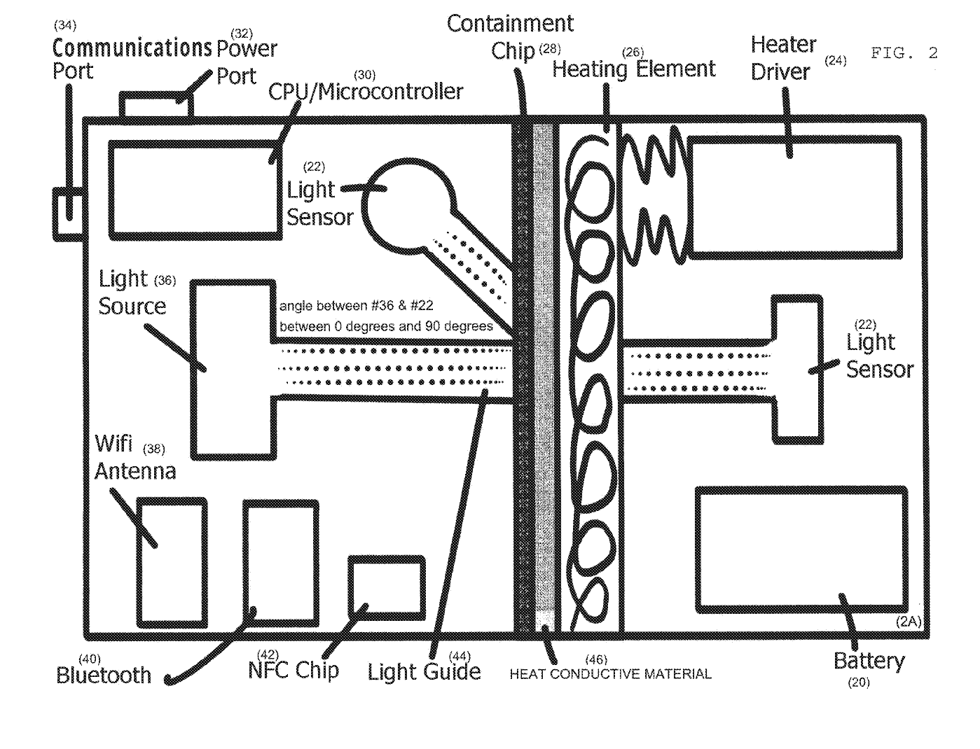

Wireless transmitter 7 can allow DNA analysis module 1A to communicate via multiple wireless protocols to any computing device whether existing or purchased and/or lab or consumer based. In some embodiments, DNA analysis module 1A may also include communications port 34 as described in greater detail with respect to FIG. 2.

Temperature control components 12 can be a logical set of components directly or indirectly responsible for both measuring the temperature of the sample as well as thermocycling the sample as per the protocol guidelines.

Temperature probe 8 can measure the temperature of the sample either indirectly or directly through contact or non-contact means. Temperature probe 8 can monitor the temperature of the sample continuously or periodically. Temperature probe 8 can be non-contact based, contact based, thermocouple, infrared, thermoresister, sonic, optical fiber.

Heating element 9 can be used to thermocycle the sample as per a testing protocol selected for the particular experiment. In some embodiments, heating element 9 can be coupled to the temperature sensing mechanism. Heating element 9 can be a direct ultrasonic/sonicator, an indirect ultrasonic/sonicator, an ultraviolet absorption based element, a magnetic induction based element, an infrared absorption based element, a microwave element, a polyester foil element, a kapton foil element, a nomex band element, a micante heating element, a ceramic element, a silicone element, a light source heatsink recycling element, a laser excitation element, a peltier/thermoelectric element, or a chemical reaction process element.

Cooling element 10 can be used to thermocycle the sample as per the requirements of the testing protocol used by the particular experiment. In some embodiments, cooling element 10 can be coupled to heating element 9 as well as to temperature probe 8. Cooling element 10 can be a peltier/thermoelectric element or a chemical reaction process.

Cooling element 10 can be optionally coupled with heating element 9. Cooling element 10 also can be optionally coupled to the temperature probe. In some embodiments, cooling element 10 and heating element 9 are separate, discrete elements.

DNA analysis module 1A can be in wireless communication via wireless transmitter 7 with an external computing unit 13. External computing unit (smartphone, computer, watch, other) can connect to the device in order to allow app to receive updates, control test portion device, enter protocol details, perform analysis of the results, download/sync test/prep protocol, scan QR code, upload new protocols, discuss results, and teach user how to operate. In at least some embodiments, module 1A has a particularly compact size when compared to existing systems, for example, on average of 8.5 cm×14.5 cm×7 cm. However, depending on the specific embodiment the size can range, for example, from 38 mm×5 mm×10 mm to 592 mm×265 mm×507 mm. In other embodiments the size can range from 6.5 cm×10.5 cm×5 cm to 9.5 cm×18.5 cm×9 cm. Module 1A can have a slit for the containment chip 5 to be placed into, and which can then slide closed, providing an optical seal preventing light from the ambient environment from penetrating into the device. Module 1A can have a power port and data ports for interfacing with an external system.

In some embodiments, DNA analysis module 1A also can include a screen 14. In some embodiments with a screen it can allow the user to see test results on the device itself. In other embodiments with a screen it can allow the user to perform basic tests without the need for an external computing unit by inputting the test protocol and seeing the results.

FIG. 2 is a block diagram of another exemplary top view layout of DNA analysis module 2A. DNA analysis module 2A can include a battery 20, one or more light sensors 22, a heater driver 24, a heating element 26, a containment chip 28, an on module CPU/microcontroller 30, a power port 32, a communication port 34, a light source 36, a Wi-Fi antenna 38, a Bluetooth communicator 40, a near field communication (“NFC”) chip 42, a light guide 44, and heat conductive material 46 to conduct heat.

Battery 20 can be, but not limited to, primary cells, secondary cells, rechargeable Alkaline, Lithium ion, Lithium polymer, Ni—Cd, Nickel metal hydride, Nickel Zinc, Lithium-air, thin film Lithium.

In certain embodiments, light sensors 22 allow for performance of a reflectance test of the sample by using a containment chip 28 with a reflective back, especially when the sample is opaque. In such embodiments, light sensor 22 is placed next to the light source, and two filters are included on the containment chip 28. One filter can be located in the path of the light source 36, and a second filter in the path of the light detector, to allow for flexibility in transmission, absorbance, and emission testing.

Heater driver 24 can be connected to heating element 26 and can be a pulse width modulated (“PWM”) N-Channel metal oxide semiconductor field effect transistor (“MOSFET”), or more generally field effect transistor (“FET”), whose voltage can be reversed when used with the embodiment that includes a cooling element. In certain embodiments, PWM can be used to control the temperature of the heater driver 24.

Sub-containment chip 300, as described in greater detail with respect to FIG. 3B, can be silicone based, silica gel based, silicon dioxide, Polydimethylsiloxane (“PDMS”), Poly(methyl methacrylate) (“PMMA”), ZEONOR® Cyclo Olefin Polymer (ZEON Chemicals L.P), conductive polymer (e.g., Clevios™, Heraeus Precious Metals GmbH & Co. KG) silicon based, and acrylic based while having the option to also be coated, covered, injected and or mixed with a material changing the optical properties into a low/high/wide/narrow/other band-pass filter.

Power port 32 can connect DNA analysis module 2A to an outside power source to provide power to DNA analysis module 2A.

Communications port 34 can be thunderbolt, USB 3.0, USB 2.0, firewire, audio/microphone, micro USB 2.0, micro USB 3.0 mini USB lightning, 30 pin apple connector, or any other proprietary or non-proprietary port.

NFC chip 42 can be a Broadcom BCM2079x or an NXP Semiconductor based NFC chip including types of active and passive NFC chips.

Light guide 44 can be connected to light source 36 and can be acrylic, fiber optic, glass, or lens based. In addition, in some embodiments, the light guide can be a prism or diffraction grating allowing the light dispersal to be measured by a CCD.

Heat conductive material 46 can conduct heat from heating element 26 to containment chip 28 according to the appropriate testing protocol.

FIG. 3A is a top view of a containment chip 302, according to embodiments of the present disclosure. Containment chip 302 can include a barcode 304, a QR code 308 and a sub-containment chip 300. The sub-containment chip 300 can include a chip well 316, a pre-made detection mixture 306, a plastic film/optical filter 310, one or more chip to microcontroller connective contacts 311, and one or more spaced electrical contacts 312. FIG. 3B is a side view of containment chip 302, showing plastic film/optical filter 310, and an optical filter 314.

In some embodiments, the containment chip is identified by a unique identifier such as a bar code, a QR code or an RFID. In some embodiments, barcode 304 identifies containment chip 302. In other embodiments the bar code 304 may be located on sub-containment chip 300, or on plastic film/optical filter 310. The barcode can include linear types such as: EAN-13, Code 128, UPC, MSI, Telepen, ITF-14.

Pre-made detection mixture 306 can be a substance that allows the DNA in the sample to replicate. Additionally, the premade detection mixture 306 attaches to the DNA during the testing sequence. The premade detection mixture 306 can come already packaged in containment chip 302. In other embodiments, pre-made detection mixture 306 can be added to a chip well 316 within sub-containment chip 300. Chip well 316 can be a recess within the sub-containment chip 300 that can hold pre-made detection mixture 306. In some embodiments the detection mixture 306 is a PCR isotherm mixture.

In some embodiments, QR code 308 identifies containment chip 302. In some embodiments the QR code may be located on the sub-containment chip 300, or on plastic film/optical filter 310. QR Codes, more generally called two-dimensional barcodes, include types such as: Aztec, Code 1, Data Matrix, High Capacity Color Barcode, EZCode, MaxiCode, PDF417, Qode, MMCC, QR Code, ShotCode, TRIPCode, SPARQCode.

Plastic film/optical filter 310 can be airtight and re-sealable. In some embodiments, plastic film/optical filter 310 can block light at specific wavelengths. In some embodiments, the plastic film/optical filter 310 can be peeled back when pre-made detection mixture 306 is added to the sub-containment chip 300 and then plastic film/optical filter 310 can be resealed. Different containment chip configurations can have different parts and the materials can be configured to either absorb or pass through light at different wavelengths. The containment chip 302 configuration and sub-containment chip 300 configuration can be dependent on the sample being tested and the test method being used.

In some embodiments microcontroller connection contacts 311 allow the containment chip 302 to connect to the processing unit, impedance detector and capacitance detector in order to measure impedance and capacitance.

In some embodiments spaced electrical contacts 312 allow the impedance and capacitance of the sample to be measured directly via microcontroller contacts 311 via containment chip 302 via process unit and impedance and capacitance detector. The spacing of the contacts will vary from different embodiments ranging from linear to exponential to logarithmic to others depending on the sample being tested on the chip and the particular embodiment. In some embodiments, an electrical current can be applied to the sample to break down cell walls and release nucleic acids from cells contained in the sample.

Optical filter 314 can consist of an acrylic, plastic, or glass covering or coating on the containment chip 302. Some embodiments can include the optical filter 314 mixed into the material the sub-containment chip 300 is made from. The optical filter 314 will filter the wavelength of light required to allow the device to perform the test. The optical filter can be a high-pass, low-pass, bandpass, narrow bandpass, or any combination thereof. In some embodiments the optical filter can be coupled with a mirror or reflective surface, allowing both reflection as well as filtering of light. As an example, in the specific case of SYBR green, a narrow bandpass filter can be used on the containment chip 302 to allow the light sensor to detect light at 520 nm.

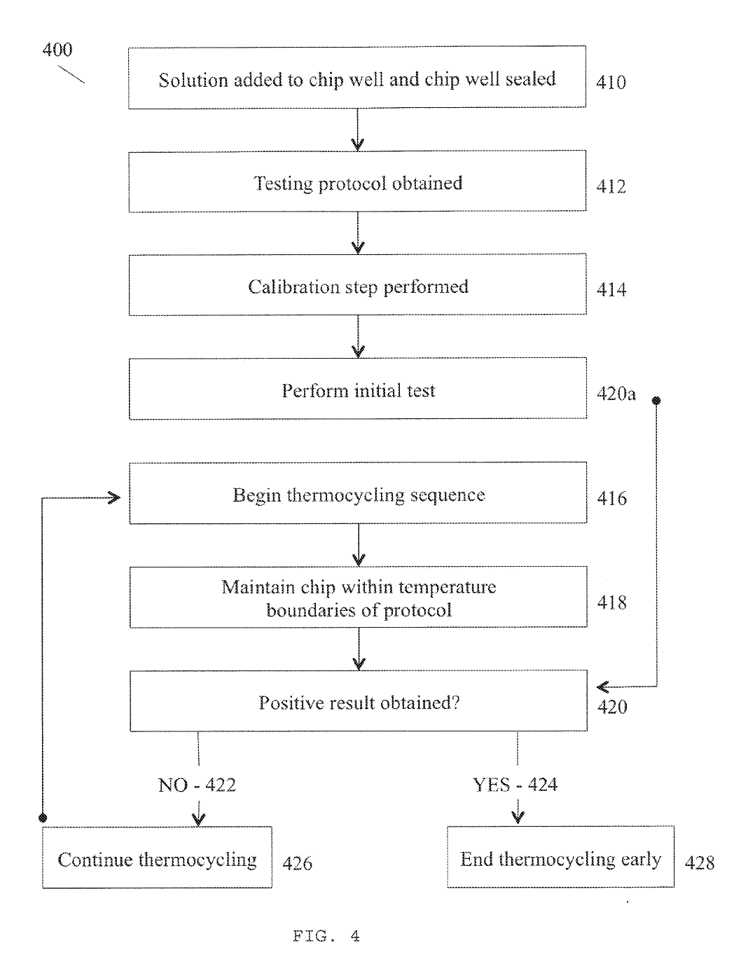

FIG. 4 is an exemplary flow chart of a process of analyzing DNA according to certain embodiments. Process for analyzing DNA 400 can include the steps of: solution added to chip well and chip well sealed 410; testing protocol obtained 412; calibration step performed 414; begin thermocycling sequence 416; maintain chip within temperature boundaries of protocol 418; check to see if positive result is obtained 420; if no 422, continue thermocycling; if yes 424; end thermocycling early 428. In certain embodiments, the sample may be detected by carrying out an initial test 420a (the same as performed at 420) prior to step 416, so that in some such embodiments the sample may be detected without having to go through any thermocycling.

Referring again to FIG. 1, a containment chip 5 is removed from the package and a sample of the solution being tested for DNA is added to the containment chip well. After the sample is added the chip is sealed. The unique identifier of the chip is either typed in or scanned into the computing unit 13 where computing unit 13 downloads the testing protocol from the Internet which is done by using the attached computing unit to access the database via included software/app. Computing unit 13 can be connected to the Internet through either a hardwire or wireless connection. Computing unit 13 interprets the testing protocol into a program via embedded software, which is then uploaded via wireless transmitter 7 to processor 6. Containment chip 5 is then inserted into DNA analysis module 1A.

Processor 6 begins the thermocycling sequence by first performing a calibration step. Light source 1 turns on and light sensor 2 takes a calibration sample, which processor 6 interprets and sets as the program's baseline. Impedance detector 3 also performs a measurement, which is used during the calibration step, which processor 6 interprets and sets as the program's baseline.

Processor 6 begins the next step in the thermocycling sequence by checking the temperature of containment chip 5 with temperature probe 8. Processor 6 uses heating element 9 and optional cooling element 10 to keep containment chip 5 temperature within the boundaries of the testing protocol that was uploaded as a program to processor 6 via wireless transmitter 7.

During each step of the PCR cycle, processor 6 uses temperature control components 12 to maintain the temperature within the protocol specifications. Also, during each step DNA detection components 4 are constantly sending impedance, absorption, and fluorescence measurements to processor 6, which compares the value to the baseline and uses an algorithm specific to the protocol to determine the frequency and presence of the DNA sequence being tested for. If processor 6 sees a positive result, the system can end the thermocycling early as per the protocol and testing parameters. If not, processor 6 continues the thermocycling process.

As DNA analysis module 1A performs the thermocycles of the protocol, processor 6 will gather the results and send those results to wireless transmitter 7. Wireless transmitter 7 sends the data to computing unit 13. Processor 6 also can have a built in algorithm that will allow further analysis of the sample and the applied process and its output to a screen 14. The software on computing unit 13 can analyze the raw data in order to come up with a meaningful result to provide to the user. The result/analysis can be completed either locally on computing unit 13 or via a third party DNA analysis service The third party service can run a more in depth analysis of the result where a local system may only be able to do an initial analysis. The service could also run the same data through an updated analysis after the fact when updates are made and notify the user automatically. This allows the computing unit to have software that is constantly updated via automated wireless means.

Those of skill in the art would appreciate that the various illustrations in the specification and drawings described herein can be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware, software, or a combination depends upon the particular application and design constraints imposed on the overall system. Skilled artisans can implement the described functionality in varying ways for each particular application. Various components and blocks can be arranged differently (for example, arranged in a different order, or partitioned in a different way) all without departing from the scope of the subject technology.

Furthermore, an implementation of the communication protocol can be realized in a centralized fashion in one computer system, or in a distributed fashion where different elements are spread across several interconnected computer systems. Any kind of computer system, or other apparatus adapted for carrying out the methods described herein, is suited to perform the functions described herein.

A typical combination of hardware and software could be a general purpose computer system with a computer program that, when being loaded and executed, controls the computer system such that it carries out the methods described herein. The methods for the communications protocol can also be embedded in a computer program product, which comprises all the features enabling the implementation of the methods described herein, and which, when loaded in a computer system is able to carry out these methods.

Computer program or application in the present context means any expression, in any language, code or notation, of a set of instructions intended to cause a system having an information processing capability to perform a particular function either directly or after either or both of the following: a) conversion to another language, code or notation; b) reproduction in a different material form. Significantly, this communication protocol can be embodied in other specific forms without departing from the spirit or essential attributes thereof.

The communications protocol has been described in detail with specific reference to these illustrated embodiments. It will be apparent, however, that various modifications and changes can be made within the spirit and scope of the disclosure as described in the foregoing specification, and such modifications and changes are to be considered equivalents and part of this disclosure.

1. A nucleic acid amplification system comprising:
a containment chip capable of holding a first sample wherein the containment chip comprises at least one reagent for amplifying a nucleic acid in the sample and an identifier for uniquely identifying the containment chip, wherein the identifier is used to select a nucleic acid amplification protocol,
a nucleic acid detection component comprising:
a light source operably linked to the containment chip, wherein the light source provides light at the wavelength necessary to perform either absorption or fluorescence measurements of the sample;
a light sensor operably linked to the containment chip, wherein the light sensor measures either absorption or fluorescence from the containment chip;
a temperature control unit operably linked to the containment chip, wherein the temperature control unit measures the temperature of the sample, and adjusts the temperature of the containment chip; and
a processor operably linked to the nucleic acid detection component and the temperature control unit, wherein the processor adjusts the light source, receives data from the light sensor, receives data from the temperature control unit, and adjusts the temperature, wherein the processer is operably connected to a database which contains the nucleic acid amplification protocol.

2. The nucleic acid amplification system of claim 1 wherein the nucleic acid detection component comprises an impedance detector operably linked to the containment chip.

3. The nucleic acid amplification system of claim 1 wherein the nucleic acid detection component comprises a capacitance detector operably linked to the containment chip.

4. The nucleic acid amplification system of claim 1 comprising a computing unit in communication with the processor.

5. The nucleic acid amplification system of claim 1 wherein the light source comprises one of a laser, a light emitting diode, an electroluminescence wire, an electroluminescence panel, a fluorescent light source, a tungsten lamp, a halogen lamp, a liquid crystal display screen, a silicon nitride lamp, a krypton lamp, a deuterium lamp, a sodium vapor lamp, a mercury vapor lamp, or a xenon light source.

6. The nucleic acid amplification system of claim 1 wherein a temperature control unit comprises one of a heating element or a cooling element.

7. The nucleic acid amplification system of claim 1 wherein the nucleic acid detection component further comprises spaced electrical contacts for applying an electrical current to the sample.

8. The nucleic acid amplification system of claim 1 wherein the containment chip further comprises an optical filter allowing the sample to both absorb as well as pass through or transmit light to the detector.

9. The nucleic acid amplification system of claim 8 wherein the containment chip comprises:
a sub-containment chip capable of holding reagents for nucleic acid amplification; and
a second optical filter.

10. The nucleic acid amplification system of claim 1 wherein the nucleic acid sample is one of a DNA sample or an RNA sample.

11. The nucleic acid amplification system of claim 1 wherein the identifier for uniquely identifying the nucleic acid sample comprises one of a bar code a QR code, or an RFID.

12. The nucleic acid amplification system of claim 1 wherein the containment chip is capable of holding a plurality of samples.

13. The nucleic acid amplification system of claim 12 wherein the containment chip further comprises a second optical filter capable of blocking light of a specific wavelength.

14. The nucleic acid amplification system of claim 12 wherein the containment chip further comprises a plastic film capable of being peeling back and resealed.

15. A method of analyzing a nucleic acid sample using a nucleic acid detection system comprising:
providing a nucleic acid sample in a containment chip wherein the containment chip comprises at least one reagent for amplifying a nucleic acid in the nucleic acid sample and an identifier for uniquely identifying the containment chip;
obtaining a nucleic acid amplification protocol from a database based on the unique identifier on the containment chip; and
performing the steps of the nucleic acid amplification protocol.

16. The method of claims 15 wherein the identifier for uniquely identifying the nucleic acid sample comprises one of a bar code, a QR code or an RFID.

17. The method of claim 15 further comprising the step of monitoring nucleic acid amplification, wherein the nucleic acid amplification protocol can be stopped once nucleic acid amplification is detected.

18. A method of analyzing nucleic acid using a nucleic acid detection system comprising:
providing a nucleic acid sample in a containment chip wherein the containment chip comprises at least one reagent for amplifying a nucleic acid in the sample and an identifier for uniquely identifying the containment chip;
obtaining a nucleic acid amplification protocol from a database based on the unique identifier on the containment chip;
performing a calibration step, comprising illuminating a light source, detecting light from a light sensor for calibration, and interpreting, by a processor in order to set a baseline,
performing an initial test to determine if a positive result is obtained by comparing to the baseline;
beginning a thermocycling sequence if a positive result is not obtained during the initial test;
maintaining the nucleic acid sample within temperature boundaries defined by the nucleic acid amplification protocol using a temperature control unit;
performing a test to determine if a positive result is obtained by comparing to the baseline;
continuing thermocycling if a positive result is not obtained during the test; and
ending thermocycling if a positive result is obtained during the test.

19. The method of claim 18 wherein the step of performing a test to determine if a positive result is obtained occurs during, outside, or in between the thermocycling process.

20. The method of claim 18 further comprising the step of electronically transmitting a request for a testing protocol corresponding to a sample to a remote server.
